(19) United States
(12) Patent Application Publication
(10) Pub. No.: US 2016/0274047 A1
(43) Pub. Date: Sep. 22, 2016

(54) BIOSENSOR STRUCTURE, METHOD OF FABRICATING THE SAME AND BIOLOGICAL DETECTION SYSTEM

(71) Applicant: BIOTECH PIONEER, KAOHSIUNG CITY (TW)

(72) Inventors: Hung-Wei WU, KAOHSIUNG CITY (TW); Yong-Han HONG, KAOHSIUNG CITY (TW); Yu-Fu CHEN, KAOHSIUNG CITY (TW); Chien-Feng LI, KAOHSIUNG CITY (TW); Hsin-Ying LEE, KAOHSIUNG CITY (TW); Pin-Wen CHEN, KAOHSIUNG CITY (TW)

(21) Appl. No.: 14/663,463

(22) Filed: Mar. 20, 2015

(51) Int. Cl. G01N 27/04; C23F 1/02; G01N 22/00; G01N 33/487

(52) U.S. Cl. CPC G01N 27/04; G01N 33/48728; C23F 1/02; G01N 22/00

(57) ABSTRACT

A biosensor structure is provided, which includes a substrate, a center conductor, a first ground conductor, a second ground conductor and a protection layer. The center conductor is disposed on the substrate and defines a detection area at the central area thereof for detection of cells or biomolecules. The first ground conductor is disposed on the substrate and is located opposite to a side of the center conductor. The second ground conductor is disposed on the substrate and is located opposite to another side of the center conductor. The protection layer is disposed on the substrate, the center conductor, the first ground conductor and the second ground conductor. In a thickness direction of the biosensor structure, the protection layer is disposed without substantially overlapping the detection area.

### BACKGROUND

1. Field of Disclosure

The invention relates to a biosensor structure, and more particularly, to a coplanar waveguide (CPW) based biosensor structure with a detection area for biodetection, a method of fabricating the biosensor structure and a biological detection system.

2. Description of Related Art

Conventional cancer detection techniques require an expensive and complex labeling process and extensive biochemical assays. Current detection methods include medical imaging and indicator analysis by using blood and urine. Although medical imaging offers highly sensitive cancer detection, it may not be validly applied to tumors that are not at least 0.1 cm in size (approximately 10⁵ tumor cells). Biomedical indicators such as prostate-specific antigen, cancer antigen 125, alpha-fetoprotein, human chorionic gonadotropin, and DR-70 have been used as tumor markers in clinical assays. However, such tumor assays have several limitations. Specifically, counting the number of cancer cells is indirect and time consuming when blood samples are separated, and furthermore, physiological conditions (infection, inflammation, and menstruation) may interfere with the accuracy of these methods. Therefore, label-free, noninvasive, nonbiological parameter detection techniques are required for current medical diagnosis applications.

The dielectric detection technique is among the most crucial tools for cellular biologists. In particular, studying signals from nonbiological parameters may reveal early signs of disease before significant changes are observed in biological signals. Currently, techniques based on optical, electrochemical, piezoelectric, and microwave-sensing approaches have been proposed. However, such techniques still have the disadvantages of restricted usage, short measurement duration and low sensitivity.

### SUMMARY

In the invention, a biosensor structure is provided for detection of object such as cells and biomolecules. By using the biosensor structure provided in the invention, electrical characteristics of the objects can be rapidly and sensitively detected. A method is also provided for fabricating the biosensor structure.

An aspect of the invention is to provide a biosensor structure. The biosensor structure includes a substrate, a center conductor, a first ground conductor, a second ground conductor and a protection layer. The center conductor is disposed on the substrate and defines a detection area at the central area thereof for detection of cells or biomolecules. The first ground conductor is disposed on the substrate and is located opposite to a side of the center conductor. The second ground conductor is disposed on the substrate and is located opposite to another side of the center conductor. The protection layer is disposed on the substrate, the center conductor, the first ground conductor and the second ground conductor. In a thickness direction of the biosensor structure, the protection layer is disposed without substantially overlapping the detection area.

In one or more embodiments, each of the center conductor, the first ground conductor and the second ground conductor comprises a first metallic layer disposed on the substrate and a second metallic layer disposed on the first metallic layer. The first metallic layer and the second metallic layer comprise different materials.

In one or more embodiments, the first metallic layer is a titanium layer, and the second metallic layer is a gold layer.

In one or more embodiments, the detection area is defined having a width of substantially between 500 μm and 2500 μm.

In one or more embodiments, the protection layer has a thickness of substantially between 35 μm and 260 μm.

In one or more embodiments, each of the center conductor, the first ground conductor and the second ground conductor has a conductivity of substantially about or greater than 10⁷ (Ω-m)⁻¹.

In one or more embodiments, the center conductor comprises a first end portion and a second end portion at two opposite ends thereof. The protection layer is disposed without covering the first end portion and the second end portion in the thickness direction of the biosensor structure.

In one or more embodiments, the center conductor has a thickness of substantially between 0.5 μm and 5 μm.

In one or more embodiments, the substrate has a conductivity of substantially less than 10 (Ω-m)⁻¹.

In one or more embodiments, the protection layer includes a polymer material.

Another aspect of the invention is to provide a method of fabricating a biosensor structure. The method includes the following steps. A substrate is provided. A center conductor, a first ground conductor and a second ground conductor are formed on the substrate, in which the center conductor is formed defining a detection area at the central area thereof for detection of cells or biomolecules, and the first ground conductor and the second ground conductor are formed being located opposite to two opposite sides of the center conductor respectively. A protection layer is formed on the substrate, the center conductor, the first ground conductor and the second ground conductor. In a thickness direction of the biosensor structure, the protection layer is formed without substantially overlapping the detection area.

In one or more embodiments, the step of forming the center conductor, the first ground conductor and the second ground conductor on the substrate includes the following steps. A first metallic layer and a second metallic layer are sequentially formed on the substrate. The first metallic layer and the second metallic layer are patterned to form the center conductor, the first ground conductor and the second ground conductor.

In one or more embodiments, the first metallic layer is a titanium layer, and the second metallic layer is a gold layer.

In one or more embodiments, the detection area is defined having a width of substantially between 500 μm and 2500 μm.

In one or more embodiments, the protection layer is formed having a thickness of substantially between 35 μm and 260 μm.

In one or more embodiments, each of the center conductor, the first ground conductor and the second ground conductor is formed having a conductivity of substantially about or greater than 10⁷ (Ω-m)⁻¹.

In one or more embodiments, the center conductor is formed having a thickness of substantially between 0.5 μm and 5 μm.

In one or more embodiments, the substrate is provided having a conductivity of substantially less than 10⁻⁵ (Ω-m)⁻¹.

Another aspect of the invention is to provide a biological detection system. The biological detection system includes a signal analyzer and a biosensor chip. The signal analyzer provides a test signal to and receives the test signal from a signal transmission path at a frequency range. The biosensor chip is coupled to the signal analyzer and located in the signal transmission path. The biosensor chip includes a substrate, a center conductor, a first ground conductor, a second ground conductor and a protection layer. The center conductor is disposed on the substrate and defines a detection area at the central area thereof for detection of cells or biomolecules. The center conductor includes a first end portion and a second end portion at two opposite ends thereof for receiving the test signal from the signal analyzer and transmitting the test signal to the signal analyzer respectively. The first ground conductor is disposed on the substrate and is located opposite to a side of the center conductor. The second ground conductor is disposed on the substrate and is located opposite to another side of the center conductor. The protection layer is disposed on the substrate, the center conductor, the first ground conductor and the second ground conductor. In a thickness direction of the biosensor structure, the protection layer is disposed without substantially overlapping the detection area.

In one or more embodiments, the frequency range is substantially between 1 GHz and 67 GHz.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1A illustrates a structure diagram biosensor structure in accordance with some embodiments of the invention;

FIG. 1B illustrates a top view of the biosensor structure shown in FIG. 1A;

FIG. 2 illustrates a simplified equivalent circuit model of single cell in suspension;

FIG. 3 illustrates an equivalent circuit model of the biosensor structure shown in FIG. 1B with cells in the defined detection area;

FIGS. 4A-4H illustrate cross-sectional diagrams of intermediate stages showing a method for fabricating a biosensor structure in accordance with some embodiments of the invention;

FIG. 5 illustrates a schematic diagram of a biological detection system in accordance with some embodiments of the invention.

FIG. 6 shows measured S21-magnitudes of the biosensor structure shown in FIG. 1A under various conditions;

FIG. 7 shows results of the measured and calculated frequency-dependent cell-based microwave attenuation of HepG2 cells at various cell densities; and

FIG. 8 shows results of the measured and calculated frequency-dependent dielectric constant of HepG2 cells at various cell densities.

### DETAILED DESCRIPTION

In the following description, the disclosure will be explained with reference to embodiments thereof. However, these embodiments are not intended to limit the disclosure to any specific environment, applications or particular implementations described in these embodiments. Therefore, the description of these embodiments is only for the purpose of illustration rather than to limit the disclosure. In the following embodiments and attached drawings, elements not directly related to the disclosure are omitted from depiction; and the dimensional relationships among individual elements in the attached drawings are illustrated only for ease of understanding, but not to limit the actual scale.

It will be understood that, although the terms “first” and “second” may be used herein to describe various elements, components, regions, layers and/or sections, these elements, components, regions, layers and/or sections should not be limited by these terms. These terms are only used to distinguish one element, component, region, layer or section from another.

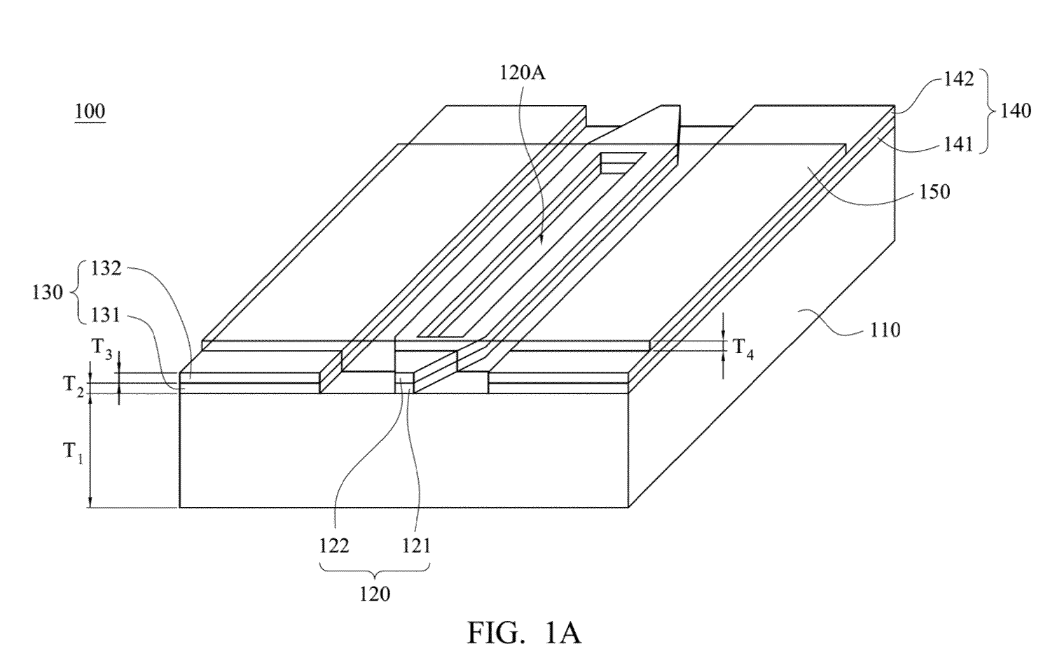

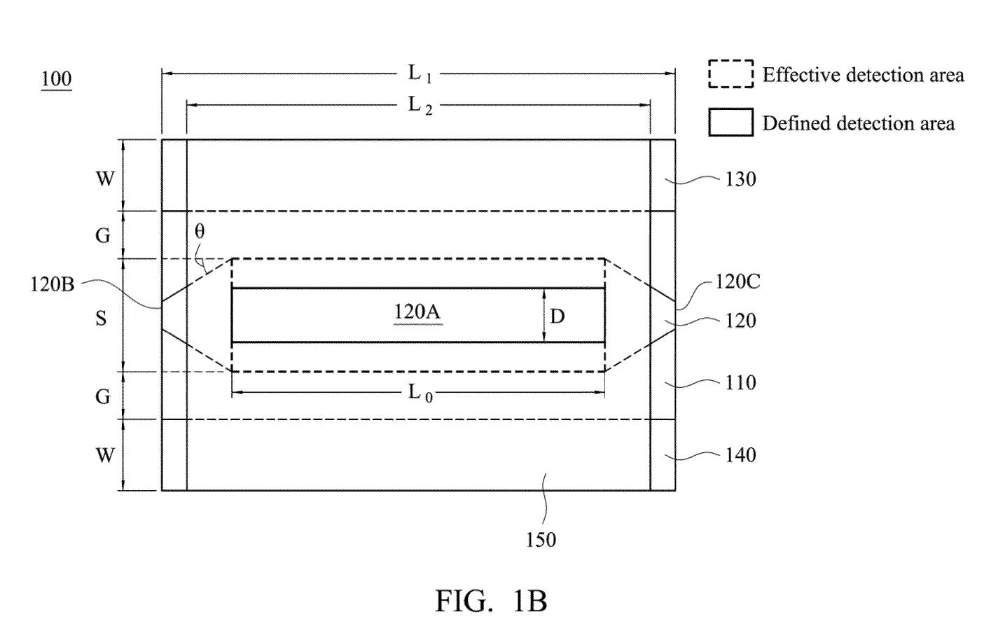

Referring to FIGS. 1A and 1B, FIG. 1A illustrates a structure diagram of a biosensor structure 100 in accordance with some embodiments of the invention, and FIG. 1B illustrates a top view of the biosensor structure 100. The biosensor structure 100 is a coplanar waveguide (CPW) based biosensor structure, which includes a substrate 110, a center conductor 120, ground conductors 130 and 140 and a protection layer 150. The substrate 110 may be a glass substrate, a ceramic substrate, a plastic substrate, a sapphire substrate, a semiconductor substrate, combinations thereof, or the like. In some embodiments, the substrate 110 has a thickness T₁ of between 500 μm and 800 μm, and the conductivity of the substrate 110 is substantially less than 10⁻⁵ (Ω-m)⁻¹. The center conductor 120 is disposed on the substrate 110. As shown in FIG. 1A, the center conductor 120 is a two-layer structure, which includes metallic layers 121 and 122. In some embodiments, the metallic layer 121 is a titanium layer, and the metallic layer 122 is a gold layer, for the center conductor 120 to have good conductivity and be easily attached to the substrate 110. In FIG. 1A, the metallic layer 121 has a thickness T₂, and the metallic layer 122 has a thickness T₃. The center conductor 120 may be alternatively a single-layer structure or a multiple-layer structure. The center conductor 120 has a conductivity of substantially about or greater than 10⁷ (Ω-m)⁻¹. The center conductor 120 has a width S and a thickness (i.e., T₂+T₃). In some embodiments, the thickness of the center conductor 120 is substantially between 0.5 μm and 5 μm.

The center conductor 120 defines a detection area 120A at the central area of the center conductor 120 for detection of objects, such as cells and/or biomolecules. The detection area 120A has a width D and a length L₀. In some embodiments, the width D of the detection area 120A is substantially between 500 μm and 2500 μm, and/or the length L₀ of the detection area 120A is substantially between 3 mm and 10 mm. In addition, the center conductor 120 includes end portions 120B and 120C at two opposite ends of the center conductor 120. The end portions 120B and 120C are used for connection with a test device, for example, a signal analyzer. The width S of the center conductor 120 tapers from one width side of the detection area 120A to one end of the center conductor 120 and from the other width side of the detection area 120A to the other end of the center conductor 120. In some embodiments, the angle θ shown in FIG. 1B is between 30 degrees and 60 degrees.

The ground conductor 130 is disposed on the substrate 110 and is located opposite to a side of the center conductor 120. As shown in FIG. 1A, the ground conductor 130 is a two-layer structure, which includes metallic layers 131 and 132. In some embodiments, the metallic layer 131 is a titanium layer, and the metallic layer 132 is a gold layer, for the ground conductor 130 to have good conductivity and be easily attached to the substrate 110. In FIG. 1A, the metallic layer 131 has the same thickness T₂ as that of the metallic layer 121, and the metallic layer 132 has the same thickness T₃ as that of the metallic layer 122. The ground conductor 130 may be alternatively a single-layer structure or a multiple-layer structure. The ground conductor 130 has a conductivity of substantially about or greater than 10⁷ (Ω-m)⁻¹. The ground conductor 130 has a width W, a length L₁ and a thickness (i.e., T₂+T₃), and is separated from the center conductor 120 by a distance G. In some embodiments, the thickness of the ground conductor 130 is substantially between 0.5 μm and 5 μm.

The ground conductor 140 is disposed on the substrate 110 and is located opposite to another side of the center conductor 120. As shown in FIG. 1A, the ground conductor 140 is also a two-layer structure, which includes metallic layers 141 and 142. In some embodiments, the metallic layer 141 is a titanium layer, and the metallic layer 142 is a gold layer, for the ground conductor 140 to have good conductivity and be easily attached to the substrate 110. In FIG. 1A, the metallic layer 141 has the same thickness T₂ as that of the metallic layer 121, and the metallic layer 142 has the same thickness T₃ as that of the metallic layer 122. The ground conductor 140 may be alternatively a single-layer structure or a multiple-layer structure. The ground conductor 140 has a conductivity of substantially about or greater than 10⁷ (Ω-m)⁻¹. The ground conductor 140 has a width W, a length L₁ and a thickness (i.e., T₂+T₃), and is separated from the center conductor 120 by a distance G. In some embodiments, the thickness of the ground conductor 140 is substantially between 0.5 μm and 5 μm.

The protection layer 150 is disposed on the substrate 110, the center conductor 120 and the ground conductors 130 and 140. The protection layer 150 is disposed to have a thickness T₄ and a length L₂ that is substantially greater than the length L₀ and substantially smaller than the length L₁. In a thickness direction of the biosensor structure 100, the protection layer 150 does not substantially overlap the detection area 120A. The protection layer 150 is disposed for concentrating objects in the detection area 120A, preventing unwanted microwave interactions with objects outside the detection area 120A of the biosensor structure 100, and avoiding short circuit in the biosensor structure 100. In some embodiments, the protection layer 150 is disposed without covering the end portions 120B and 120C in the thickness direction of the biosensor structure 100. The protection layer 150 may be a SU-8 photoresist layer, a polydimethylsiloxane (PDMS) layer, a polymethylmethacrylate (PMMA) photoresist layer, a JSR photoresist layer, or the like. In some embodiments, the protection layer 150 includes a polymer material, and the thickness T₄ of the protection layer 150 is substantially between 35 μm and 260 μm.

As shown in FIG. 1B, the biosensor structure 100 includes a defined detection area and an effective detection area. The defined detection area is the area of the detection area 120A, which is designed to locate objects to be detected in areas where the electromagnetic (EM) field is strongly concentrated. The objects to be detected may be cells, biomolecules, etc. In some embodiments, some objects located in the effective detection area may be valid and can be verified by a simulator, such as 3D full-wave EM simulator.

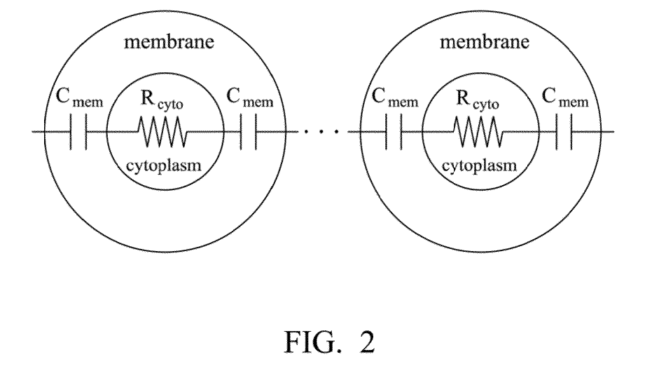

In the case of cells, FIG. 2 illustrates a simplified equivalent circuit model of a single cell in suspension. The cell cytoplasm is a highly conductive ionic solution with a large concentration of dissolved organic material that forms a resistive pathway to the electrical signal in the electrical equivalent of the system. The cell membrane consists of a thin phospholipid bilayer with extremely low conductivity. The thin phospholipid bilayer acts as a dielectric material offering a capacitive pathway to the system. A single cell is analogous to a cytoplasm resistor R_{cyto} in series with a membrane capacitor Cₘₑₘ.

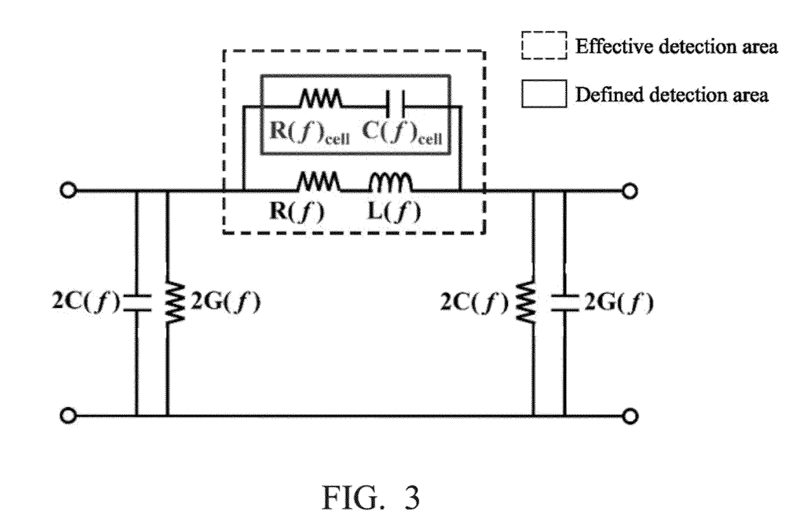

FIG. 3 illustrates an equivalent circuit model of the biosensor structure 100 shown in FIG. 1B with cells in the detection area. Based on the description of FIG. 2, all of the cells located in the detection area are modeled as a series circuit containing the frequency-dependent cell-based resistance R(f)_{cell} and capacitance C(f)_{cell}; R(f)_{cell} and C(f)_{cell} represent the total magnitude of the cells. The circuit model shown in FIG. 3 includes frequency-dependent resistance R(f), inductance L(f), conductance G(f) and capacitance C(f) of the biosensor structure 100.

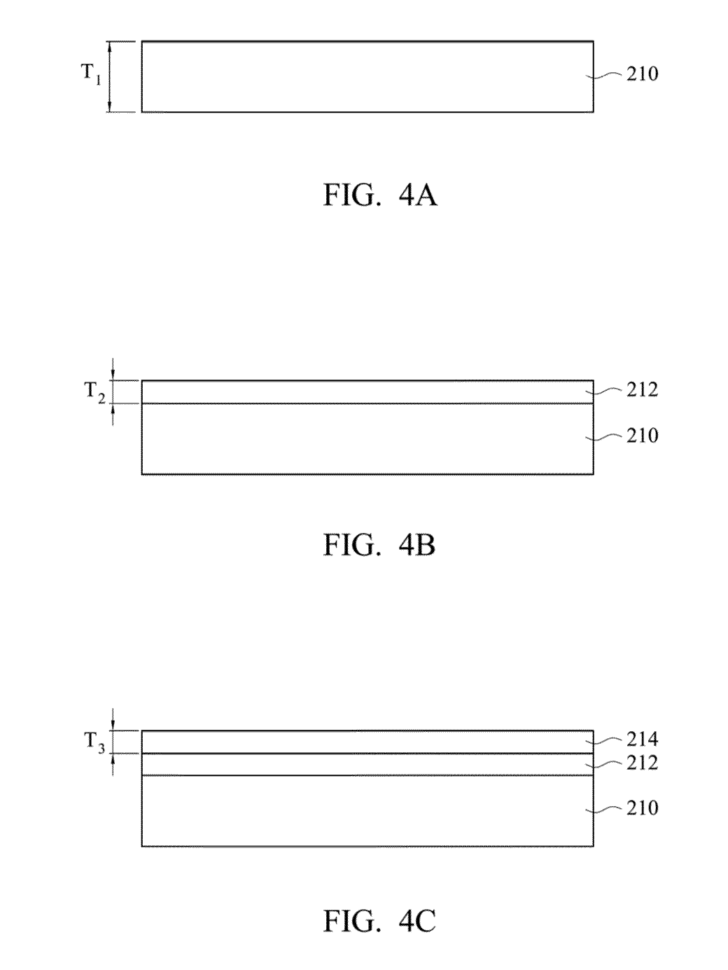

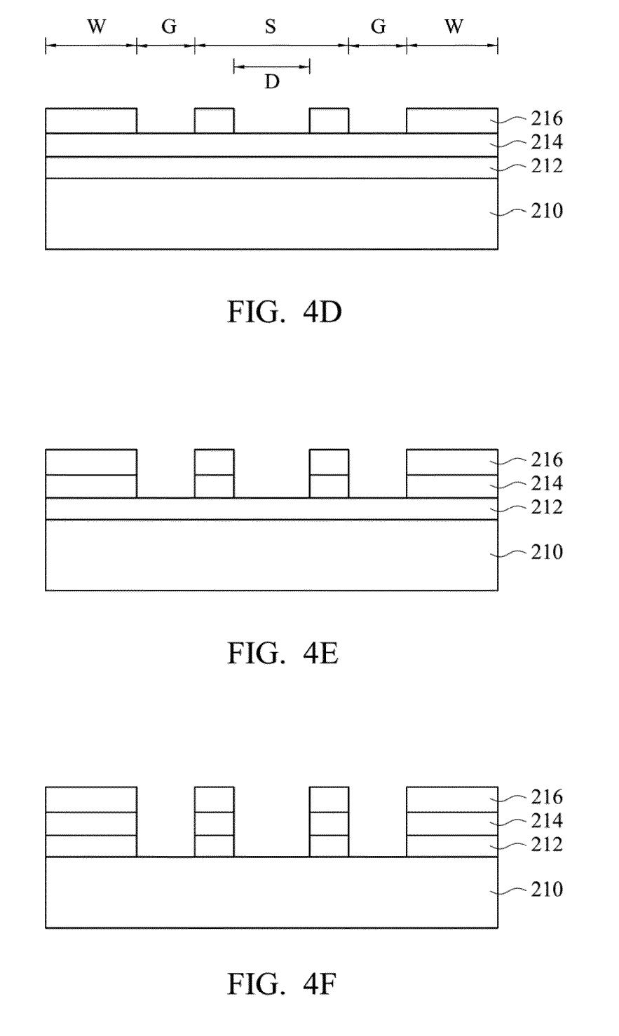

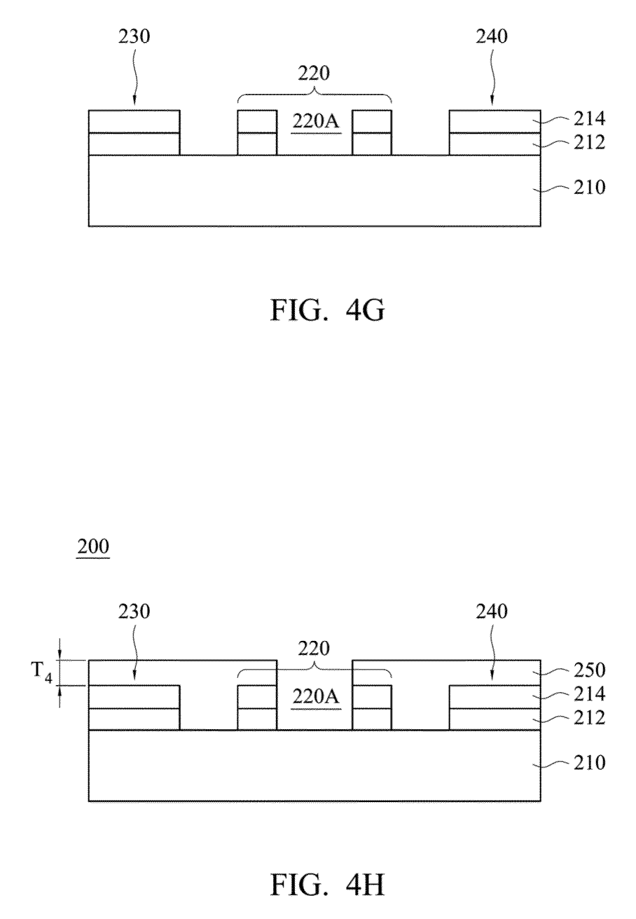

Referring to FIGS. 4A-4H, FIGS. 4A-4H illustrate cross-sectional diagrams of intermediate stages showing a method for fabricating a biosensor structure 200 in accordance with some embodiments of the invention.

As shown in FIG. 4A, a substrate 210 is provided. The substrate 210 may be a glass substrate, a ceramic substrate, a plastic substrate, a sapphire substrate, a semiconductor substrate, combinations thereof, or the like. In some embodiments, the substrate 210 is provided having a thickness T₁ of between 500 μm and 800 μm and a conductivity of substantially less than 10⁻⁵ (Ω-m)⁻¹.

As shown in FIG. 48, a metallic layer 212 is formed on the substrate 210. The metallic layer 212 is formed having a conductivity of substantially about or greater than 10⁷ (Ω-m)⁻¹ and a thickness T₂. The metallic layer 212 may be formed of any suitable metal, and may be formed by a deposition process such as chemical vapor deposition (CVD), low-pressure CVD (LPCVD), metal organic CVD (MOCVD), plasma enhanced CVD (PECVD), physical vapor deposition (PVD), atomic layer deposition (ALD), and molecular beam epitaxy (MBE), or a sputtering process such as radio frequency (RF) magnetron sputtering and direct-current (DC) magnetron sputtering, but is not limited thereto. In some embodiments, the metallic layer 212 is a titanium layer, and the metallic layer 212 is formed by an RF magnetron process using a titanium metal target with a purity of 99.9995%. Before forming the metallic layer 212 on the substrate 210, a universal standard RCA cleaning process may be performed for cleaning the substrate 210. Furthermore, the substrate 210 may be cleaned in acetone, rinsed in deionized water and subsequently dried in flowing nitrogen gas.

As shown in FIG. 4C, a metallic layer 214 is formed on the metallic layer 212. The metallic layer 214 is formed having a conductivity of substantially about or greater than 10⁷ (Ω-m)⁻¹ and a thickness T₃. The metallic layer 214 may be formed of any suitable metal, and may be formed by a deposition process such as CVD, LPCVD, MOCVD, PECVD, PVD, ALD, and MBE, or a sputtering process such as RF magnetron sputtering and DC magnetron sputtering, but is not limited thereto. In some embodiments, the metallic layer 214 is a gold layer, and the metallic layer 214 is formed by an e-beam evaporation process using a gold chip with a purity of 99.999%. In some embodiments, the summation of the thicknesses T₂ and T₃ is substantially between 0.5 μm and 5 μm.

As shown in FIG. 4D, a patterned photoresist layer 216 is formed on the metallic layer 214. The patterned photoresist layer 216 is used as a mask for the following etching steps. The patterned photoresist layer 216 defines the widths W, S, D, and the distance G, as shown in FIG. 4D. In some embodiments, the width D is defined substantially between 500 μm and 2500 μm.

As shown in FIG. 4E, portions of the metallic layer 214 corresponding to the width D and the distance G are removed by an etching process. The etching process to the metallic layer 214 may be performed by using dry etching technology, wet etching technology, combinations thereof, or the like.

As shown in FIG. 4F, portions of the metallic layer 212 corresponding to the width D and the distance G are removed by an etching process. The etching process to the metallic layer 212 may be performed by using dry etching technology, wet etching technology, combinations thereof, or the like. In some embodiments, the metallic layers 212 and 214 are etched during the same etching process.

As shown in FIG. 4G, after the etching process, the patterned photoresist layer 216 is stripped. The remained portions of the metallic layers 212 and 214 form a center conductor 220 and ground conductors 230 and 240, and the area corresponding to the width D is formed as a detection area 220A.

As shown in FIG. 4H, a protection layer 250 is formed on the substrate 210, the center conductor 220 and the ground conductors 230 and 240, such that the biosensor structure 200 is formed. The protection layer 250 fills the gap between the center conductor 220 and the ground conductor 230/240 and does not substantially overlap the detection area 220A in a thickness direction of the biosensor structure 200. The protection layer 250 may be formed of SU-8 photoresist, polydimethylsiloxane, polymethylmethacrylate, JSR photoresist, or the like. The protection layer 250 may be formed by performing a deposition process such as high density plasma CVD (HDPCVD), a spin-on metal (SOM) process, or any other suitable process. In some embodiments, the protection layer 250 is formed including a polymer material and having the thickness T₄ substantially between 35 μm and 260 μm.

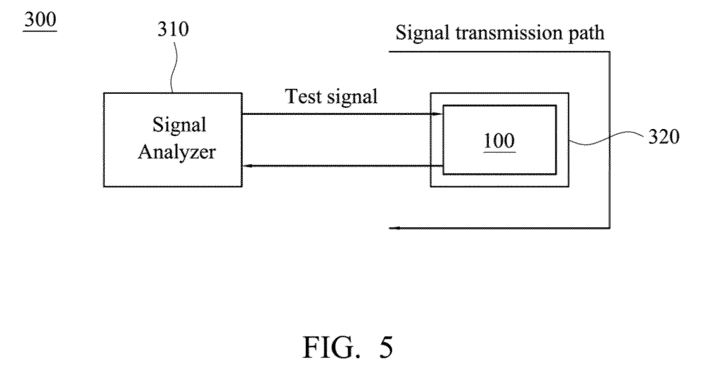

Referring to FIG. 5, FIG. 5 illustrates a schematic diagram of a biological detection system 300 in accordance with some embodiments of the invention. As shown in FIG. 5, the biological detection system 300 includes a signal analyzer 310 and a biosensor chip 320. The signal analyzer 310 is configured to provide a test signal to the biosensor chip 320 and receive the test signal from a signal transmission path at a frequency range. The biosensor chip 320 is coupled to the signal analyzer 310 and is located in the signal transmission path. The biosensor chip 320 includes the biosensor structure 100 shown in FIG. 1A. One of the end portions 120B and 120C is coupled to the signal analyzer 310 for receiving the test signal from the signal analyzer 310, while the other of the end portions 120B and 120C coupled to the signal analyzer 310 for transmitting the test signal to the signal analyzer 310. In some embodiments, the signal analyzer 310 provides the test signal at a frequency range of between 1 GHz and 67 GHz.

In the following experiments, human hepatoma (HepG2) cells are used, and the characteristics of the biological detection system 300 are listed as below. For the substrate 110, the thickness T₁ is 700 μm, the relative dielectric constant is 5.27 Fm⁻¹, and the loss tangent is 0.003. For the center conductor 120 and the ground conductors 130 and 140, the material of the metallic layers 121, 131 and 141 is titanium, the material of the metallic layers 122, 132 and 142 is gold, the thickness T₂ is 1.5 μm, the thickness T₃ is 0.5 μm, the width S is 1160 μm, the width D is 600 μm, the length L₁ is 6600 μm, the width W is 900 μm, and the distance G is 25 μm. For the protection layer 150, the material of the protection layer is a SU-8 photoresist, the thickness T₄ is 55 μm, and the length L₂ is 3000 μm. For the signal analyzer 310, the frequency range of the test signal ranges from 1 GHz to 40 GHz. In various embodiments, the signal analyzer 310 may generate the test signal with another frequency range, for example, from 1 GHz to 67 GHz.

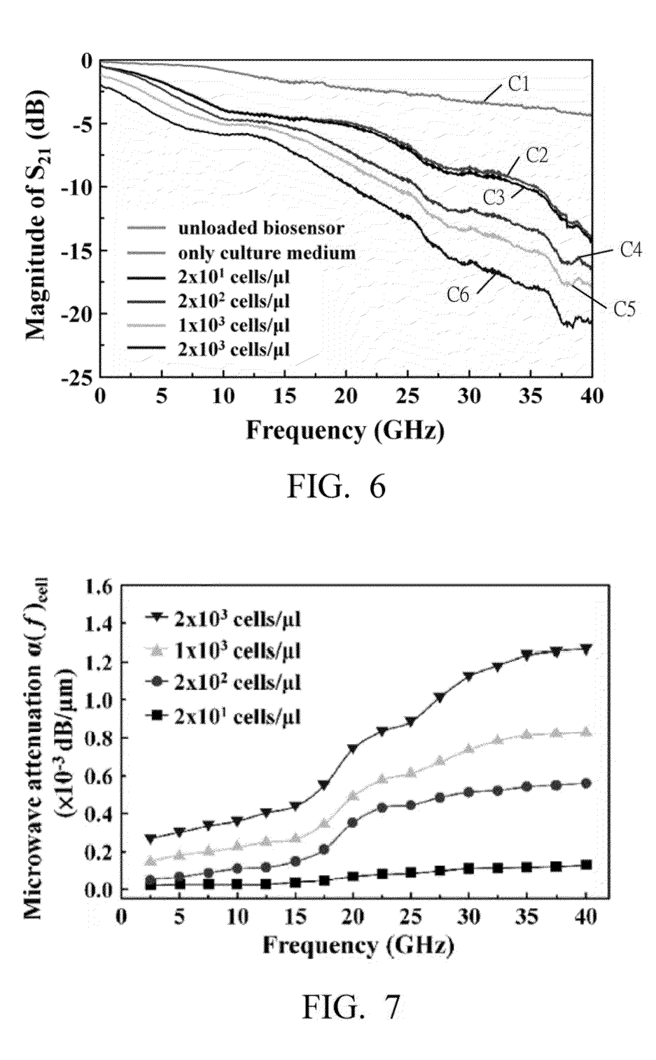

FIG. 6 shows measured S21-magnitudes of the biosensor structure 100 under various conditions. In FIG. 6, curves C1-C6 are illustrated. The curve C1 represents that the biosensor structure 100 is unloaded, that is, no cultured medium (with or without the cells) is put into the detection area 120A. The curve C2 represents that only cultured medium (i.e., cell density of 0) is put into the detection area 120A. The curve C3 represents that the cultured medium with cell density of 2×10¹ cells/μL is put into the detection area 120A. The curve C4 represents that the cultured medium with cell density of 2×10² cells/μL is put into the detection area 120A. The curve C5 represents that the cultured medium with cell density of 1×10³ cells/μL is put into the detection area 120A. The curve C6 represents that the cultured medium with cell density of 2×10³ cells/μL is put into the detection area 120A. As shown in FIG. 6, when the cultured medium (with or without the cells) is put into the detection area 120A, electromagnetic waves penetrate the cultured medium and/or the cells, causing microwave attenuation and S21-magnitude degradation. The microwave parasitic effects can be divided into the cultured medium and substrate materials. Also, as shown in FIG. 6, the S21-magnitude degrades as the cell density changes from 0 (i.e., only cultured medium) to 2×10³ cells/μL. Cells can be considered electric charges remaining within a homogeneous dielectric material system. Therefore, S21-magnitude degradation at different cell densities is associated with polarization effects (including ion vibration and deformation losses) between cells at microwave frequencies. As can be seen from FIG. 6, the lower detection limit of the biosensor structure 100 is approximately 20 cells/μL.

By using the biological detection system 300, four calibrated scattering parameters (S-parameters; S11, S12, S21 and S22) measured on the biosensor structure 100 can be obtained, and the frequency-dependent propagation constant (γ(f)=α(f)+jβ(f)) can be derived from the eigenvalues of the transmission matrix (i.e., ABCD matrix), where α(f) is the microwave attenuation and β(f) is related to the wave number of the eigenvalues. The frequency-dependent cell-based microwave attenuation α(f)_{cell} of the cells can be obtained by Equation (1):

$${α ( f )}_{cell} = 8.686 \cdot [ - \frac{1}{{L}_{1}} Re \{ {ln [ \frac{1 - {S}_{11}^{2} + {S}_{21}^{2}}{2 {S}_{21}} \pm {[ \frac{{( {S}_{11}^{2} - {S}_{21}^{2} + 1 )}^{2} - {( 2 {S}_{11} )}^{2}}{{( 2 {S}_{11} )}^{2}} ]}^{1 \text{/} 2} ]}^{- 1} \} ] . \qquad ( 1 )$$

The biosensor structure 100 is designed on a dielectric substrate of finite thickness. The method can be applied to any single-layer CPW line-based biosensor structure with quasi-TEM propagation.

Referring back to FIG. 3, the frequency-dependent resistance R(f), inductance L(f), conductance G(f) and capacitance C(f) are derived from γ(f)×Z₀(f)=R(f)+jωL(f) and γ(f)×Z₀(f)=G(f)+jωC(f), where R(f)=Re[γ(f)×Z₀(f)]_{unloaded}, L(f)=Im[γ(f)×Z₀(f)]_{unloaded}/ω, G(f)=Re[γ(f)/Z₀(f)]_{unloaded} and C(f)=Im[γ(f)/Z₀(f)]_{unloaded}/ω, where ω is the angular frequency and Z₀(f) is the characteristic impedance of the biosensor structure 100. The values of the frequency-dependent resistance R(f), inductance L(f), conductance G(f) and capacitance C(f) are summarized in Table 1. In Table 1, the frequency-dependent resistance R(f) represents the ohmic loss in the biosensor structure 100. Because the application of an electric field does not change the magnetic flux penetration in the dielectric media, the extracted frequency-dependent inductance L(f) exhibits nearly identical values of 0.188 pH/μm, and the frequency-dependent conductance G(f) exhibits a behavior similar to that of the frequency-dependent resistance R(f). Specifically, the frequency-dependent conductance G(f) exhibits higher values at higher frequencies than at lower frequencies. Variation in the frequency-dependent conductance G(f) is affected by the polarization current in the substrate 110 and the uniformity and quality of the substrate 110. Moreover, the frequency-dependent capacitance C(f) results from the polarization mechanisms of the substrate 110 at microwave frequencies and is sufficiently small to reduce the crosstalk and power consumption associated with the biosensor structure 100.

TABLE 1

| Frequency | R(f) | L(f) | G(f) | C(f) |
|---|---|---|---|---|
| (GHz) | (mΩ/μm) | (pH/μm) | (μS/μm) | (fF/μm) |
| 5 | 1.456 | 0.188 | 0.0019 | 0.248 |
| 10 | 3.456 | 0.188 | 0.0045 | 0.243 |
| 15 | 7.416 | 0.188 | 0.0093 | 0.239 |
| 20 | 9.719 | 0.188 | 0.012 | 0.236 |
| 25 | 11.470 | 0.188 | 0.0151 | 0.230 |
| 30 | 13.781 | 0.188 | 0.0175 | 0.225 |
| 35 | 16.4 | 0.188 | 0.0196 | 0.221 |
| 40 | 18.510 | 0.188 | 0.0243 | 0.217 |

In FIG. 3, the frequency-dependent cell-based resistance R(f)_{cell} and capacitance C(f)_{cell} by a transform to ABCD parameters of two-port circuit can be obtained by Equation (2):

$${[ \begin{matrix}{S}_{11} & {S}_{12} \\ {S}_{21} & {S}_{22}\end{matrix} ]}_{cell} ⇔ {[ \begin{matrix}A & B \\ C & D\end{matrix} ]}_{cell} , \qquad ( 2 )$$

where B is expressed by Equation (3):

$$B = {Z}_{0} ( f ) \frac{( 1 + {S}_{11} ) ( 1 + {S}_{22} ) - {S}_{12} {S}_{21}}{2 {S}_{21}} = \frac{1}{{Y}_{2}} . \qquad ( 3 )$$

In FIG. 3, the frequency-dependent cell-based resistance R(f)_{cell} and capacitance C(f)_{cell} can be obtained from Equation (3) as R(f)_{cell}=1/Re[Y₂] and C(f)_{cell}=Im[Y₂], where

$$\begin{matrix}{Y}_{2} = \frac{1}{R ( f ) + j ω L ( f )} {\vert}_{unloaded} + \frac{j ω {R ( f )}_{cell} \cdot {C ( f )}_{cell} + 1}{j ω {C ( f )}_{cell}} {\vert}_{cell} \\ = \frac{\begin{matrix}( 1 - {ω}^{2} L ( f ) \cdot {C ( f )}_{cell} ) + j ( ω \cdot {R ( f )}_{cell} \cdot \\ C {( f )}_{cell} + ω \cdot R ( f ) \cdot {C ( f )}_{cell} )\end{matrix}}{\begin{matrix}( R ( f ) - {ω}^{2} {R ( f )}_{cell} \cdot {C ( f )}_{cell} ) + j ( ω \cdot R ( f ) \cdot \\ R {( f )}_{cell} {C ( f )}_{cell} + ω \cdot L ( f ) )\end{matrix}}\end{matrix} . \qquad ( 4 )$$

For Equation (4), the values of frequency-dependent resistance R(f) and capacitance C(f) for the biosensor structure 100 can be found in the aforementioned paragraphs. To evaluate the effects of the RF power treatment on the biosensor structure 100, a simplified electrical circuit model can be applied on the association with a frequency-dependent cell-based resistance R(f)_{cell} and a capacitance C(f)_{cell} to describe the electrical properties of the cells.

FIG. 7 shows the measured and calculated frequency-dependent cell-based microwave attenuation α(f)_{cell} of HepG2 cells at various cell densities. As the cell density of the HepG2 cells increases from 2×10¹ cells/IL to 2×10³ cells/μL, the frequency-dependent cell-based microwave attenuation α(f)_{cell} surpasses that of the unloaded biosensor structure 100. The frequency-dependent cell-based microwave attenuation α(f)_{cell} indicates the microwave attenuation of the HepG2 cells without microwave parasitic effects. After the cell density of the HepG2 cells increases, the frequency-dependent cell-based microwave attenuation α(f)_{cell} is 0.12×10⁻³ dB/μm for the cell density of 2×10¹ cells/μL, 0.58×10⁻³ dB/μm for the cell density of 2×10² cells/μL, 0.81×10⁻³ dB/μm for the cell density of 1×10³ cells/μL, and 1.26×10⁻³ dB/μm for the cell density of 2×10³ cells/μL at 40 GHz, respectively. The variation of the frequency-dependent cell-based microwave attenuation α(f)_{cell} by cell density is caused by the polarization current in the HepG2 cells. Additionally, the frequency-dependent cell-based microwave attenuation α(f)_{cell} is also related to the dielectric loss of the HepG2 cells described by loss tangent tan [δ(f)]_{cell}. The average values of the loss tangent tan [δ(f)]_{cell} at different cell densities are 0.021 (2×10¹ cells/μL), 0.032 (2×10² cells/μL), 0.056 (1×10³ cells/μL) and 0.102 (2×10³ cells/μL), respectively. Such results are useful because considering dielectric loss is crucial in biomedical diagnosis.

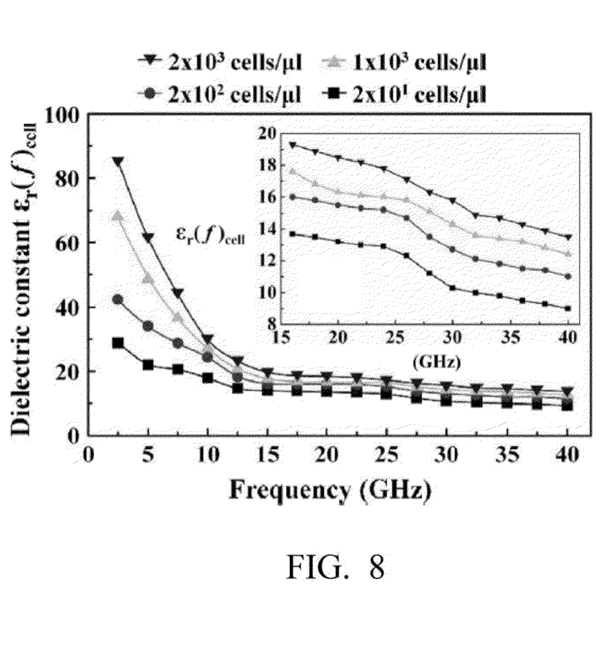

FIG. 8 shows the frequency-dependent cell-based dielectric constant ∈ᵣ(f)_{cell} of HepG2 cells at various cell densities. As the cell density of the HepG2 cells increases, the frequency-dependent cell-based dielectric constant ∈ᵣ(f)_{cell} shows an average of 11.37 for the cell density of 2×10¹ cells/μL, 13.58 for the cell density of 2×10² cells/μL, 14.6 for the cell density of 1×10³ cells/μL, and 16.4 for the cell density of 2×10³ cells/μL from 15 GHz to 40 GHz, respectively. The frequency-dependent cell-based dielectric constant ∈ᵣ(f)_{cell} is associated with the permittivity of the HepG2 cells by polarization effects within a range of between 1 GHz and 40 GHz. The frequency-dependent cell-based dielectric constant ∈ᵣ(f)_{cell} is the highest at lower frequencies and decreases indistinct and consecutive plateaus as the frequency increases. The gamma dispersions (≧10⁹ to 10¹¹ Hz) play a central role in polarization effects, which are caused by ion vibration, deformation, and the aqueous content of the HepG2 cells. The polarization of the HepG2 cells can be expressed approximately as the relationship between the polarizability and the EM field of the HepG2 cells as Equation (5):

$$P = {\sum }_{j} {N}_{j} {α}_{j} E ( j ) , \qquad ( 5 )$$

where Nⱼ is the cell density at cell site j, αⱼ is the polarizability of the HepG2 cells at cell site j, and E(j) is the EM field at cell site j. The frequency-dependent cell-based microwave attenuation α(f)_{cell} and the frequency-dependent cell-based dielectric constant ∈ᵣ(f)_{cell} of the HepG2 cells are dominated by the cell density. The results show that the biological detection system with a CPW based biosensor structure of the invention successfully performs dielectric detection of cells.

To sum up, the biosensor structure of the invention can provide a wider bandwidth for high-sensitivity detection. The biosensor structure of the invention is designed for label-free detection and effective measurement of frequency-dependent parameters (e.g., microwave attenuation and dielectric constant) of objects (such as cells and/or biomolecules) at various object densities. The microwave parasitic effects can be eliminated using the biological detection system of the invention. The sensitivity of the biosensor structure is associated with the object density of the objects, making it an effective tool for detecting the object density rapidly, even when the object density is extremely low. The biosensor structure of the invention can be widely used for the dielectric characterization of any type of cells and/or biomolecules.

Although the disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims.

What is claimed is:

1. A biosensor structure comprising:
a substrate;
a center conductor disposed on the substrate, the center conductor defining a detection area at the central area thereof for detection of cells or biomolecules;
a first ground conductor disposed on the substrate and located opposite to a side of the center conductor;
a second ground conductor disposed on the substrate and located opposite to another side of the center conductor; and
a protection layer disposed on the substrate, the center conductor, the first ground conductor and the second ground conductor;
wherein, in a thickness direction of the biosensor structure, the protection layer is disposed without substantially overlapping the detection area.

2. The biosensor structure of claim 1, wherein each of the center conductor, the first ground conductor and the second ground conductor comprises a first metallic layer disposed on the substrate and a second metallic layer disposed on the first metallic layer, wherein the first metallic layer and the second metallic layer comprise different materials.

3. The biosensor structure of claim 2, wherein the first metallic layer is a titanium layer, and the second metallic layer is a gold layer.

4. The biosensor structure of claim 1, wherein the detection area is defined having a width of substantially between 500 μm and 2500 μm.

5. The biosensor structure of claim 1, wherein the protection layer has a thickness of substantially between 35 μm and 260 μm.

6. The biosensor structure of claim 1, wherein each of the center conductor, the first ground conductor and the second ground conductor has a conductivity of substantially about or greater than 10⁷ (Ω-m)⁻¹.

7. The biosensor structure of claim 1, wherein the center conductor comprises a first end portion and a second end portion at two opposite ends thereof, wherein the protection layer is disposed without covering the first end portion and the second end portion in the thickness direction of the biosensor structure.

8. The biosensor structure of claim 1, wherein the center conductor has a thickness of substantially between 0.5 μm and 5 μm.

9. The biosensor structure of claim 1, wherein the substrate has a conductivity of substantially less than 10⁻⁵ (Ω-m)⁻¹.

10. The biosensor structure of claim 1, wherein the protection layer comprises a polymer material.

11. A method of fabricating a biosensor structure, the method comprising:
providing a substrate;
forming a center conductor, a first ground conductor and a second ground conductor on the substrate, wherein the center conductor is formed defining a detection area at the central area thereof for detection of cells or biomolecules, and wherein the first ground conductor and the second ground conductor are formed being located opposite to two opposite sides of the center conductor respectively; and
forming a protection layer on the substrate, the center conductor, the first ground conductor and the second ground conductor;
wherein, in a thickness direction of the biosensor structure, the protection layer is formed without substantially overlapping the detection area.

12. The method of claim 11, wherein forming the center conductor, the first ground conductor and the second ground conductor on the substrate comprises:
forming a first metallic layer and a second metallic layer sequentially on the substrate; and
patterning the first metallic layer and the second metallic layer to form the center conductor, the first ground conductor and the second ground conductor.

13. The method of claim 12, wherein the first metallic layer is a titanium layer, and the second metallic layer is a gold layer.

14. The method of claim 11, wherein the detection area is defined having a width of substantially between 500 μm and 2500 μm.

15. The method of claim 11, wherein the protection layer is formed having a thickness of substantially between 35 μm and 260 μm.

16. The method of claim 11, wherein each of the center conductor, the first ground conductor and the second ground conductor is formed having a conductivity of substantially about or greater than 10⁷ (Ω-m)⁻¹.

17. The method of claim 11, wherein the center conductor is formed having a thickness of substantially between 0.5 μm and 5 μm.

18. The method of claim 11, wherein the substrate is provided having a conductivity of substantially less than 10⁻⁵ (Ω-m)⁻¹.

19. A biological detection system, comprising:
a signal analyzer for providing a test signal to and receiving the test signal from a signal transmission path at a frequency range; and
a biosensor chip coupled to the signal analyzer and located in the signal transmission path, the biosensor chip comprising:
a substrate;
a center conductor disposed on the substrate, the center conductor defining a detection area at the central area thereof for detection of cells or biomolecules, and the center conductor comprising a first end portion and a second end portion at two opposite ends thereof for receiving the test signal from the signal analyzer and transmitting the test signal to the signal analyzer respectively;
a first ground conductor disposed on the substrate and located opposite to a side of the center conductor;
a second ground conductor disposed on the substrate and located opposite to another side of the center conductor; and
a protection layer disposed on the substrate, the center conductor, the first ground conductor and the second ground conductor;
wherein, in a thickness direction of the biosensor chip, the protection layer is disposed without substantially overlapping the detection area.

20. The biological detection system of claim 19, wherein the frequency range is substantially of between 1 GHz and 67 GHz.
